(19) United States
(12) Patent Application Publication
(10) Pub. No.: US 2022/0072077 A1
(43) Pub. Date: Mar. 10, 2022

(54) COMPOSITION FOR PREVENTING OR TREATING SKIN DISEASES COMPRISING BRIDALWREATH SPIREA

(71) Applicant: Hongcheon Institute of Medicinal Herb, Gangwon-do (KR)

(72) Inventors: Yongjun Lee, Gangwon-do (KR); Young Han Kim, Gangwon-do (KR)

(21) Appl. No.: 17/417,992

(22) PCT Filed: Dec. 18, 2019
(86) PCT No.: PCT/KR2019/018040

§ 371 Date: Oct. 14, 2021

(30) Foreign Application Priority Data
Dec. 27, 2018 (KR) 10-2018-0170129

(51) Int. Cl. A61K 36/73; A61P 17/06

(52) U.S. Cl. CPC A61K 36/73; A61K 2236/333; A61P 17/06

(57) ABSTRACT

The present invention relates to a pharmaceutical composition for prevention or treatment of skin disease which contains Spiraea prunifolia var. simpliciflora extract or a fraction thereof, a method of treating skin disease which includes administering the pharmaceutical composition to an individual, a food composition for improvement of skin disease which contains Spiraea prunifolia var. simpliciflora extract or a fraction thereof, a quasi-drug composition for prevention or improvement of skin disease which contains Spiraea prunifolia var. simpliciflora extract or a fraction thereof, and a cosmetic composition for prevention or improvement of skin disease which contains Spiraea prunifolia var. simpliciflora extract or a fraction thereof. It has been confirmed that the Spiraea prunifolia var. simpliciflora extract of the present invention can effectively treat skin disease such as psoriasis without causing side effects and thus may be widely used for the prevention or treatment of various skin diseases.

### TECHNICAL FIELD

The present invention relates to a composition for prevention or treatment of skin disease which contains Spiraea prunifolia var. simpliciflora extract. More specifically, the present invention relates to a pharmaceutical composition for prevention or treatment of skin disease which contains Spiraea prunifolia var. simpliciflora extract or a fraction thereof, a method of treating skin disease which includes administering the pharmaceutical composition to an individual, a food composition for improvement of skin disease which contains Spiraea prunifolia var. simpliciflora extract or a fraction thereof, a quasi-drug composition for prevention or improvement of skin disease which contains Spiraea prunifolia var. simpliciflora extract or a fraction thereof, and a cosmetic composition for prevention or improvement of skin disease which contains Spiraea prunifolia var. simpliciflora extract or a fraction thereof.

### BACKGROUND ART

The skin is gradually damaged by intrinsic factors such as aging or external factors such as ultraviolet rays, external pollutants, and stress. As the function to protect the skin from these factors is weakened, the ability to protect and proliferate cells is diminished. In this case, the regeneration process with respect to damaged skin is initiated in the individual. This skin regeneration process is a response by the skin tissue to the damage, and continues for 2 days to 3 weeks after the skin is damaged.

Meanwhile, the problem of skin damage caused by external environmental factors has recently increased, and a representative environmental factor that causes such skin damage is particulate matter, which has recently seen a sharp increase. Depending on the particle size, dust is divided into total suspended particles (TSP) having a particle size of 50 μm or less and particulate matter (PM) having a significantly small particle size, and particulate matter is further divided into fine particles having a diameter of less than 10 μm (PM10) and ultrafine particles having a diameter of less than 2.5 μm (PM_{2.5}). When particulate matter enters the body, immune cells act to remove and discharge the particulate matter, and this action may be accompanied by inflammation as a side effect thereof. Particulate matter penetrates not only into the respiratory organs, such as the respiratory tract, bronchi, and lungs, but also into the pores of the skin, and it stimulates the skin to cause trouble and inflammation and further to exacerbate the skin condition, and particulate matter may further adversely affect chronic skin disease such as atopic dermatitis.

Steroids that are most widely used as a traditional therapeutic agent for skin disease cause side effects such as diabetes and high blood pressure, and the misuse and abuse thereof has become a social problem. In the case of immunomodulators that have been recently developed, it has been reported that antibody preparations, which have been expected to safely cause immunosuppressive action by acting only on a specific part as the target, cause unexpected serious side effects such as immune disorders, infections, and cancer. Therefore, in order to develop a preparation which can effectively treat skin disease without causing side effects, studies using natural products have been actively conducted. For example, Korean Patent Application Laid-Open No. 2010-0080972 discloses a native grass extract obtained using a fermented tree sap that can be used for alleviation and improvement of skin disease, and Korean Patent Application Laid-Open No. 2011-0086894 discloses a composition for external application to skin, which contains calcined powder of shell or waste coral and an alkaline calcium solution extracted from calcined powder of shell or waste coral. However, preparations developed using natural products as described above do not cause side effects, but have a disadvantage in that the therapeutic effect is insufficient, and there is an urgent need for supplementation.

### DISCLOSURE

### Technical Problem

With this background, the present inventors intensively conducted studies to develop a preparation that can effectively treat skin disease without causing side effects, as a result, they have found that Spiraea prunifolia var. simpliciflora extract exhibits an effect of treating skin disease without causing side effects, thereby completing the present invention.

### Technical Solution

An object of the present invention is to provide a pharmaceutical composition for prevention or treatment of skin disease which contains Spiraea prunifolia var. simpliciflora extract or a fraction thereof.

Another object of the present invention is to provide a method of treating skin disease which includes administering the pharmaceutical composition to an individual.

Still another object of the present invention is to provide a food composition for improvement of skin disease which contains Spiraea prunifolia var. simpliciflora extract or a fraction thereof.

Still another object of the present invention is to provide a quasi-drug composition for prevention or improvement of skin disease which contains Spiraea prunifolia var. simpliciflora extract or a fraction thereof.

Still another object of the present invention is to provide a cosmetic composition for prevention or improvement of skin disease which contains Spiraea prunifolia var. simpliciflora extract or a fraction thereof.

### Advantageous Effects

It has been confirmed that the Spiraea prunifolia var. simpliciflora extract of the present invention can effectively treat skin disease such as psoriasis without causing side effects and may thus be widely used for the prevention or treatment of various skin diseases.

### BRIEF DESCRIPTION OF DRAWINGS

FIG. 1 is a graph illustrating the analysis results of the cytotoxicity of Spiraea prunifolia var. simpliciflora extract;

FIG. 2 is a graph illustrating the results acquired by comparing changes in the amount of IL-6 produced depending on the concentration of Spiraea prunifolia var. simpliciflora extract treated;

FIG. 3 is a photograph illustrating the Western blot analysis results showing the results acquired by comparing changes in the phosphorylation level of Stat3 protein depending on the concentration of Spiraea prunifolia var. simpliciflora extract treated;

FIG. 4 is a schematic diagram illustrating the experimental process for verifying the effect of Spiraea prunifolia var. simpliciflora extract using an animal model of skin disease;

FIG. 5 is a graph illustrating changes in the body weight of Balb/c mice treated with imiquimod (IMQ), Spiraea prunifolia var. simpliciflora extract (SPS), or Dermovate (CLO);

FIG. 6 is a photograph illustrating changes in the appearance of ears of Balb/c mice treated with imiquimod (IMQ), Spiraea prunifolia var. simpliciflora extract (SPS), or Dermovate (CLO) depending on the passage of breeding time;

FIG. 7 is a graph illustrating changes in the thickness of ear skin of Balb/c mice treated with imiquimod (IMQ), Spiraea prunifolia var. simpliciflora extract (SPS), or Dermovate (CLO) depending on the passage of breeding time;

FIG. 8 is a graph illustrating changes in the level of erythema of ear skin of Balb/c mice treated with imiquimod (IMQ), Spiraea prunifolia var. simpliciflora extract (SPS), or Dermovate (CLO) depending on the passage of breeding time;

FIG. 9 is an optical micrograph illustrating the results acquired by staining the skin tissue of Balb/c mice treated with imiquimod (IMQ), Spiraea prunifolia var. simpliciflora extract (SPS), or Dermovate (CLO) with hematoxylin and eosin; and

FIG. 10 is a graph illustrating the quantitative analysis results of the epidermal thickness of the skin tissue of Balb/c mice treated with imiquimod (IMQ), Spiraea prunifolia var. simpliciflora extract (SPS), or Dermovate (CLO).

### DETAILED DESCRIPTION OF THE INVENTION

An embodiment of the present invention for achieving the objects provides a pharmaceutical composition for prevention or treatment of skin disease which contains Spiraea prunifolia var. simpliciflora extract or a fraction thereof.

Another embodiment of the present invention provides the use of Spiraea prunifolia var. simpliciflora extract or a fraction thereof for the prevention or treatment of skin disease.

As used herein, the term “Spiraea prunifolia var. simpliciflora” refers to a kind of deciduous shrub growing in the slopes or rocky areas at the edge of forests, and is distinguished from other plants belonging to the genus Spiraea in Korea since it has 4 to 5 flowers blooming on a short branch. “Spiraea prunifolia var. simpliciflora” is an ornamental plant that is planted in gardens, parks, and roadsides, and is also called Orixa japonica Thunb. or Hieracium umbellatum L. “Spiraea prunifolia var. simpliciflora” is called “fried millet tree” since its flower shape looks like fried millet grains glued together. “Spiraea prunifolia var. simpliciflora” is also planted for hedges, its young leaves are edible, and its roots are medicinal.

In the present invention, as the Spiraea prunifolia var. simpliciflora, commercially available products may be purchased and used, or those collected from nature or grown may be used, but the Spiraea prunifolia var. simpliciflora is not limited thereto.

As used herein, the term “extract” refers to a liquid ingredient obtained by immersing a target substance in various solvents and then extracting the target substance at room temperature or in a heated state for a certain period of time, or a product such as a solid obtained by removing the solvent from the liquid ingredient. The term “extract” may be construed comprehensively to include, in addition to the products, a diluted solution of the product, a concentrate thereof, an adjusted product thereof, a purified product, and the like. Accordingly, the Spiraea prunifolia var. simpliciflora extract provided in the present invention may be construed to include the extract itself and extracts in all formulations that can be formed using the extract, such as an extract obtained by extracting the flowers, leaves, fruits, stems, roots, bark, sap, and the like of Spiraea prunifolia var. simpliciflora, a diluted solution or concentrate of the extract, a dried product obtained by drying the extract, an adjusted product or purified product of the extract, or any mixture of these.

In the extract of Spiraea prunifolia var. simpliciflora of the present invention, the method for extracting the mixture is not particularly limited, and the mixture may be extracted by way of a method commonly used in the art. Non-limiting examples of the extraction method include hot water extraction, ultrasonic extraction, filtration, and reflux extraction. These may be performed singly or in a combination of two or more methods.

In the present invention, the kind of solvent used for the extraction is not particularly limited, and an arbitrary solvent known in the art may be used. Non-limiting examples of the extraction solvent include water, alcohol, or a mixed solvent thereof. These may be used singly or in mixture of one or more, and specifically water may be used. When an alcohol is used as a solvent, specifically an alcohol having 1 to 4 carbon atoms may be used.

As used herein, the term “fraction” refers to a product obtained by performing fractionation in order to separate a specific ingredient or a group of specific ingredients from a mixture containing various constituents.

The fractionation method for obtaining the fraction in the present invention is not particularly limited, and fractionation may be performed by way of a method commonly used in the art. Non-limiting examples of the fractionation method include a solvent fractionation method in which fractionation is performed by treating the mixture with various solvents, ultrafiltration fractionation in which fractionation is performed by allowing the mixture to pass through an ultrafiltration membrane having a constant molecular weight cut-off value, chromatographic fractionation in which various types of chromatography (those designed for separation depending on the size, charge, hydrophobicity, or affinity) are performed, and any combination thereof. Specifically, there may be mentioned a method in which a fraction is obtained from the extract obtained by extracting Spiraea prunifolia var. simpliciflora of the present invention by treating the extract with a predetermined solvent.

In the present invention, the kind of fractionation solvent used to obtain the fraction is not particularly limited, and an arbitrary solvent known in the art may be used. Non-limiting examples of the fractionation solvent include polar solvents such as water and an alcohol having 1 to 4 carbon atoms; non-polar solvents such as hexane, ethyl acetate, chloroform, and dichloromethane; or any mixed solvent thereof. These may be used singly or in a mixture of one or more, but the fractionation solvent is not limited thereto.

The extract or fraction may be prepared in the form of a dry powder after extraction and used, but is not limited thereto.

As used herein, the term “skin disease” refers to a disease in which abnormal symptoms such as inflammation, erythema, thickening, fibrosis, and keratin appear on the skin due to factors such as genetic factors, physiological factors, and environmental factors.

In the present invention, the skin disease is not particularly limited thereto as long as the symptoms can be prevented, improved, alleviated, or treated by way of the treatment with Spiraea prunifolia var. simpliciflora extract or a fraction thereof. As an example, the skin disease may be an inflammatory skin disease induced by excessive secretion of inflammatory cytokines. As other examples, the skin disease may be atopic dermatitis that is a type of inflammatory skin disease induced by excessive secretion of IL-6, allergic dermatitis, psoriasis, seborrheic dermatitis, contact dermatitis, lupus erythematosus, purulent acne, and papular urticaria and the like.

As used herein, the term “prevention” refers to all actions in which skin disease is inhibited or delayed by administration of a composition containing the extract.

As used herein, the term “treatment” refers to all actions in which the symptoms of skin disease are improved or beneficially changed by administration of a composition containing the extract.

The pharmaceutical composition of the present invention may contain the extract at 0.001% to 80% by weight, specifically 0.001% to 70% by weight, more specifically 0.001% to 60% by weight with respect to the total weight of the composition, but is not limited thereto.

The pharmaceutical composition may further contain a pharmaceutically acceptable carrier, excipient, or diluent commonly used in the preparation of pharmaceutical compositions, and the carrier may include a carrier which does not occur naturally. Examples of the carrier, excipient and diluent include lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, and mineral oil.

The pharmaceutical composition may be formulated in the form of a tablet, a pill, a powder, granules, a capsule, a suspension, an internal solution, an emulsion, a syrup, a sterilized aqueous solution, a non-aqueous solution, a suspension, an emulsion, a freeze-dried preparation, a transdermal absorbent, a gel, a lotion, an ointment, a cream, a patch, a cataplasma, a paste, a spray, a skin emulsion, a skin suspension, a transdermal delivery patch, a drug-containing bandage, or a suppository, each by way of a conventional method, and used. Specifically, in the case of being formulated, the formulation may be prepared using a commonly used diluent or excipient such as a filler, a weight agent, a binder, a wetting agent, a disintegrant, or a surfactant. Solid preparations for oral administration include, but are not limited to, a tablet, a pill, a powder, granules, a capsule, and the like. Such solid preparations may be prepared by mixing at least one or more excipients, for example, starch, calcium carbonate, sucrose, lactose, and gelatin. In addition to simple excipients, lubricants such as magnesium stearate and talc may also be used. Such solid preparations may be prepared by adding various excipients, for example, a wetting agent, a sweetening agent, a fragrance, and a preserving agent in addition to liquids and liquid paraffin for oral use. Preparations for parenteral administration include a sterilized aqueous solution, a non-aqueous solution, a suspension, an emulsion, a freeze-dried preparation, and a suppository. As the non-aqueous solvent and suspending agent, propylene glycol, polyethylene glycol, and vegetable oil such as olive oil, injectable esters such as ethyl oleate, and the like may be used. As the base of suppositories, Witepsol, Macrogol, Tween 61, cacao butter, laurin, glycerogelatin, and the like may be used.

Another embodiment of the present invention provides a method of treating skin disease which includes administering the pharmaceutical composition described above to an individual suspected of having a skin disease.

As used herein, the term “administration” refers to the action in which a composition containing the extract is introduced into an individual by way of an appropriate method.

As used herein, the term “individual” refers to all animals, such as rats, mice, and livestock, including humans, that have or can develop skin disease. As a specific example, the term “individual” may refer to a mammal including a human.

The pharmaceutical composition of the present invention is administered in a pharmaceutically effective amount. The term “pharmaceutically effective amount” refers to an amount sufficient to treat a disease with a reasonable benefit/risk ratio applicable to medical treatment, and the effective dose level may be determined depending on factors including kind of individual and severity, age, gender, drug activity, sensitivity to drug, time of administration, route of administration and excretion rate, duration of treatment, and concomitant drugs and other factors well known in the medical art. For example, the Spiraea prunifolia var. simpliciflora extract may be administered in a dose of 0.01 mg/kg to 500 mg/kg, specifically 10 mg/kg to 100 mg/kg per day, and the administration may be performed one time or several times a day.

The pharmaceutical composition may be administered as an individual therapeutic agent or may be administered in combination with other therapeutic agents, and may be administered sequentially or simultaneously with conventional therapeutic agents. The pharmaceutical composition may be administered in a single or multiple manner. It is important to administer the pharmaceutical composition in an amount so that the maximum effect can be acquired with a minimum amount without side effects in consideration of all of the above factors, and the amount can be easily determined by those skilled in the art.

The pharmaceutical composition may be administered orally or parenterally (for example, intravenously, subcutaneously, intraperitoneally, or topically) depending on the intended method. The dosage varies depending on the condition and weight of the patient, the severity of the disease, the drug form, and the route and time of administration, but may be appropriately selected by those skilled in the art.

Still another embodiment of the present invention provides a food composition for improvement of skin disease which contains Spiraea prunifolia var. simpliciflora extract or a fraction thereof.

Here, the definitions of the Spiraea prunifolia var. simpliciflora, extract, fraction, and skin disease are as described above.

As used herein, the term “improvement” refers to all actions in which a parameter related to a condition to be treated, for example, the severity of symptoms, is at least diminished by administration of a composition containing the extract.

As used herein, the “food” includes meat, sausage, bread, chocolate, candies, snacks, confectionery, pizza, ramen, other noodles, gums, dairy products including ice cream, various soups, beverages, teas, drinks, alcoholic beverages, vitamin complexes, functional foods, health foods, and the like, and includes all foods in a conventional sense.

The food composition of the present invention is derived from Spiraea prunifolia var. simpliciflora that can be ingested on a daily basis, and can be thus expected to have a high skin disease improvement effect, and can be significantly usefully utilized for health promotion purposes.

The functional food is the same term as food for special health use (FoSHU), and refers to food that is processed so as to efficiently exhibit bioregulatory functions in addition to nutrition supply and thus have high medical and medical treatment effects. Here, the term “function(al)” means to acquire useful effects for health use such as regulation of nutrients or physiological action on the structure and function of the human body. The food of the present invention may be prepared by way of a method commonly used in the art, and the food may be prepared by adding raw materials and ingredients commonly added in the art at the time of the preparation. The food may be prepared in any formulation without limitation as long as it is a formulation recognized as food.

The composition for food of the present invention may be prepared in various types of formulations, is prepared using natural substances as raw materials unlike general drugs, and thus has the advantage of not causing side effects that may occur during long-term administration of drugs, and exhibits excellent portability. Therefore, the food of the present invention can be ingested as an adjuvant to enhance the skin disease improvement effect.

The health food refers to food having an active health maintenance or promotion effect as compared to general food, and the health supplement food refers to food for the purpose of health supplementation. In some cases, the terms functional food, health food, and health supplement food are used interchangeably.

Specifically, the functional food is food prepared by adding the Spiraea prunifolia var. simpliciflora extract or a fraction thereof of the present invention to food materials such as beverages, teas, spices, gums, and confectionery or by encapsulation, powdering, and suspension of the Spiraea prunifolia var. simpliciflora extract or a fraction thereof of the present invention, is meant to exert a specific effect on health when ingested, and is prepared using food as a raw material unlike general drugs to have the advantage of not causing side effects that may occur during long-term administration of drugs.

The food composition may further contain a physiologically acceptable carrier, and the kind of carrier is not particularly limited, and any carrier commonly used in the art may be used.

The food composition may contain additional ingredients commonly used in food compositions to improve odor, taste, visual appearance, and the like. The food composition may contain, for example, vitamins A, C, D, E, B1, B2, B6, and B12, niacin, biotin, folate, pantothenic acid, and the like. The food composition may contain minerals such as zinc (Zn), iron (Fe), calcium (Ca), chromium (Cr), magnesium (Mg), manganese (Mn), copper (Cu), and chromium (Cr); and amino acids such as lysine, tryptophan, cysteine, and valine.

The food composition may contain food additives such as preservatives (potassium sorbate, sodium benzoate, salicylic acid, sodium dehydroacetate, and the like), disinfectants (bleaching powder and high bleaching powder, sodium hypochlorite, and the like), antioxidants (butylhydroxyanisole (BHA), butylhydroxytoluene (BHT), and the like), colorants (tar pigments and the like), coloring agents (sodium nitrite, sodium nitrite, and the like), a bleaching agent (sodium sulfite), seasoning (monosodium glutamate (MSG) and the like), sweeteners (dulcin, cyclamate, sodium saccharin, and the like), flavoring (vanillin, lactones, and the like), leavening agents (alum, potassium hydrogen D-tartrate, and the like), a fortifying agent, an emulsifying agent, a thickening agent (texture modifier), a coating agent, a gum base agent, a foam inhibitor, a solvent, and an improving agent. The additives may be selected depending on the kind of food and used in appropriate amounts.

An example of the food composition of the present invention may be a health drink composition, and in this case, the health drink composition may contain various flavor agents or natural carbohydrates as an additional ingredient like a conventional drink. The above-mentioned natural carbohydrates may be monosaccharides such as glucose and fructose; disaccharides such as maltose and sucrose; polysaccharides such as dextrin and cyclodextrin; and sugar alcohols such as xylitol, sorbitol, and erythritol. As the sweetening agents, natural sweetening agents such as thaumatin, stevia extract; synthetic sweetening agents such as saccharin and aspartame; and the like may be used. The ratio of the natural carbohydrates may be generally about 0.01 g to 0.04 g, specifically about 0.02 g to 0.03 g per 100 mL of the health drink composition of the present invention.

In addition to the above, the health drink composition may contain various nutrients, vitamins, electrolytes, flavoring agents, colorants, pectic acid, salts of pectic acid, alginic acid, salts of alginic acid, organic acids, protective colloidal thickening agents, pH adjusting agents, stabilizers, preservatives, glycerin, alcohols or carbonating agents, and the like. In addition to these, the health drink composition may contain the pulp for the production of natural fruit juice, fruit juice beverage, or vegetable beverage. These ingredients may be used independently or in a mixture. The ratio of these additives is not significantly important, but is generally selected in a range of 0.01 to 0.1 parts by weight per 100 parts by weight of the health drink composition of the present invention.

Still another embodiment of the present invention provides a quasi-drug composition for prevention or improvement of skin disease which contains Spiraea prunifolia var. simpliciflora extract or a fraction thereof.

Still another embodiment of the present invention provides the use of Spiraea prunifolia var. simpliciflora extract or a fraction thereof for the prevention or improvement of skin disease.

Here, the definitions of the Spiraea prunifolia var. simpliciflora, extract, fraction, skin disease, prevention, and improvement are as described above.

As used herein, the term “quasi-drug” refers to fibers or rubber products used for the purpose of treating, alleviating, curing, or preventing diseases of humans or animals or those similar thereto; articles that have a weak action on the human body or do not act directly on the human body, and are not instruments or machines, and those similar thereto; and articles corresponding to one of the preparations used for sterilization and deinsectization to prevent infection and purposes similar thereto, from which those other than instruments, machines, or devices among articles used for the purpose of diagnosing, treating, alleviating, curing, or preventing diseases of humans or animals and those other than instruments, machines, or devices used for the purpose of pharmacologically affecting the structure and function of humans or animals, and also includes external preparations for skin and personal care products.

When the Spiraea prunifolia var. simpliciflora extract of the present invention is added to a quasi-drug composition for purpose of preventing or improving skin disease, the extract may be added as it is or used together with other quasi-drug ingredients, and may be appropriately used according to a conventional method. The amount of the active ingredient mixed may be suitably determined depending on the purpose of use (prophylactic, health, or therapeutic treatment).

The quasi-drug composition includes, but is not particularly limited to, personal care products, external preparations for skin, disinfectant cleaners, a shower foam, wet tissues, a detergent soap, a hand wash, a mask, or an ointment. While not particularly limited thereto, the external preparations for skin specifically may be prepared in the form of an ointment, a lotion, a spray, a patch, a cream, a powder, a suspension, a gel product, or a gel and used. While not particularly limited thereto, the personal care products specifically may be a soap, wet tissues, tissues, a shampoo, a toothpaste, hair care products, an air freshener gel, or a cleaning gel.

Still another embodiment of the present invention provides a cosmetic composition for prevention or improvement of skin disease which contains Spiraea prunifolia var. simpliciflora extract or a fraction thereof.

Here, the definitions of the Spiraea prunifolia var. simpliciflora, extract, fraction, skin disease, prevention and improvement are as described above.

As in the pharmaceutical composition, the Spiraea prunifolia var. simpliciflora extract may be contained at a proportion of 0.1% to 20% by weight with respect to the total weight of the composition. Specifically, the Spiraea prunifolia var. simpliciflora extract is contained at a proportion of 0.1% to 5% by weight, more specifically 0.4% to 0.6% by weight, still more specifically 0.5% by weight with respect to the total weight of the composition, but is not limited thereto.

The ingredients contained in the cosmetic composition of the present invention include ingredients commonly used in cosmetic compositions in addition to the extract. For example, the cosmetic composition may further contain one or more additives selected from the group consisting of water, a surfactant, a moisturizer, a lower alcohol, a chelating agent, a bactericide, an antioxidant, a preservative, a coloring, and a fragrance.

The cosmetic composition may be prepared in any conventionally prepared formulation, and may be formulated into, for example, a solution, an emulsion, a suspension, a paste, a cream, a lotion, a gel, a powder, a spray, a surfactant-containing cleansing agent, an oil, a soap, a liquid detergent, a bath agent, a foundation, a makeup base, an essence, a skin toner, a foam, a pack, a softening water, a sunscreen cream, or a sun oil, but is not limited thereto.

When the formulation of the present invention is a solution or an emulsion, a solvent, a solubilizer, or an emulsifier is used as a carrier ingredient, and examples thereof include water, ethanol, isopropanol, ethyl carbonate, ethyl acetate, benzyl alcohol, benzyl benzoate, propylene glycol, 1,3-butyl glycol oil, glycerol aliphatic esters, and fatty acid esters of polyethylene glycol or sorbitan.

When the formulation of the present invention is a suspension, a liquid diluent such as water, ethanol, or propylene glycol, suspending agents such as ethoxylated isostearyl alcohol, polyoxyethylene sorbitol esters, and polyoxyethylene sorbitan esters, microcrystalline cellulose, aluminum metahydroxide, bentonite, tragacanth, or the like may be used as a carrier ingredient.

When the formulation of the present invention is a paste, a cream, or a gel, animal oil, vegetable oil, wax, paraffin, starch, tragacanth, cellulose derivatives, polyethylene glycol, silicone, bentonite, silica, talc, zinc oxide, or the like may be used as a carrier ingredient.

When the formulation of the present invention is a powder or a spray, lactose, talc, silica, aluminum hydroxide, calcium silicate, or polyamide powder may be used as a carrier ingredient. In particular, when the formulation of the present invention is a spray, propellants such as chlorofluorohydrocarbons, propane/butane, or dimethyl ether may be additionally contained.

When the formulation of the present invention is a surfactant-containing cleansing agent, aliphatic alcohol sulfates, aliphatic alcohol ether sulfates, sulfosuccinic acid monoesters, isethionates, imidazolinium derivatives, methyl taurate, sarcosinates, fatty acid amide ether sulfates, alkylamido betaines, aliphatic alcohols, fatty acid glycerides, fatty acid diethanolamides, vegetable oil, lanolin derivatives, ethoxylated glycerol fatty acid esters, or the like may be used as a carrier ingredient.

The ingredients contained in the cosmetic composition may be contained in amounts generally used in the field of dermatology.

### EXAMPLES

Hereinafter, the present invention will be described in more detail with reference to Examples. However, these Examples are for illustrative purposes only, and the scope of the present invention is not intended to be limited by these Examples.

### Example 1

### Analysis of Cytotoxicity of Spiraea prunifolia var. simpliciflora Extract

The cytotoxicity of the Spiraea prunifolia var. simpliciflora extract was analyzed using a sample obtained by dissolving a methyl alcohol extract of Spiraea prunifolia var. simpliciflora purchased from the Korea Plant Extract Bank in DMSO.

Roughly, a cell line of cultured human-derived skin keratinocytes that could simulate the symptoms of skin psoriasis was treated with the Spiraea prunifolia var. simpliciflora extract, and the changes in cell viability were measured through the WST 1 assay.

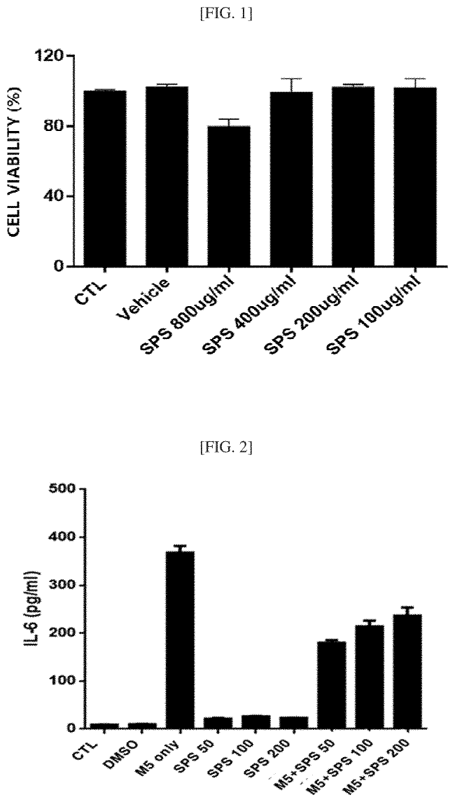

Specifically, the HaCaT cell line, which is a human-derived skin keratinocyte, was cultured under conditions of 37° C. and 5% CO₂ using DMEM (Dulbecco's modified Eagle Medium) medium containing 10% (v/v) fetal bovine serum (FBS, Gibco) and antibiotic antimycotic. When the cells had grown to about 80% of the 100 mm culture dish, the cells were lightly washed with PBS for cell detachment, then treated with trypsin-EDTA (Gibco/BRL), then left at 37° C. for 8 minutes, and collected. Subculture was performed one time every 2 to 3 days. The collected cells were seeded in a 96-well plate at a concentration of 3×10⁴ cells/mL, pre-cultured in an incubator for 24 hours while maintaining the conditions of 37° C. and 5% CO₂, then treated with the Spiraea prunifolia var. simpliciflora extract at various concentrations (100 μg/mL to 800 μg/mL), and cultured for 24 hours. Thereafter, the cultured cells were treated with water-soluble tetrazolium salt (WST-1) and cultured for 1 hour so that WST-1 was reduced by the enzymatic action of viable cells. Thereafter, measurement was performed at 450 nm using a microplate reader (FIG. 1). Here, cells that were not treated with the Spiraea prunifolia var. simpliciflora extract were used as a negative control, and cells treated only with DMSO were used as a positive control.

FIG. 1 is a graph illustrating the analysis results of the cytotoxicity of Spiraea prunifolia var. simpliciflora extract.

As illustrated in FIG. 1, it was confirmed that the cell viability was diminished by about 10% in the case of treating the cells with the Spiraea prunifolia var. simpliciflora extract at a concentration of 800 μg/mL, but cytotoxicity was not observed when the cells were treated with the Spiraea prunifolia var. simpliciflora extract at concentrations less than 800 μg/mL.

### Example 2

### Analysis of Effect of Spiraea prunifolia var. simpliciflora Extract on IL-6 Production

As IL-6, which is a kind of cytokine, is known as a substance to cause skin disease through an inflammatory response, it was attempted to determine whether the Spiraea prunifolia var. simpliciflora extract affects the production of IL-6.

Roughly, HaCaT cells were treated with M5 (IL22, I1a, TNF-α, Oncostatin M, IL17A; J Immunol 2010, 184:5263-5270) to increase the amount of IL-6 produced and then treated with the Spiraea prunifolia var. simpliciflora extract at various concentrations (50 μg/mL, 100 μg/mL, or 200 μg/mL) to measure changes in the amount of IL-6 produced (FIG. 2). Here, cells that were not treated with the Spiraea prunifolia var. simpliciflora extract were used as a negative control, cells treated only with M5 were used as a positive control, and cells that were not treated with M5 but were treated only with the Spiraea prunifolia var. simpliciflora extract at various concentrations were used as a control.

FIG. 2 is a graph illustrating the results acquired by comparing changes in the amount of IL-6 produced depending on the concentration of Spiraea prunifolia var. simpliciflora extract treated.

As illustrated in FIG. 2, it was confirmed that the amount of IL-6 produced significantly increased when the HaCaT cells were treated with M5, but the amount of IL-6 produced increased by the treatment with M5 was diminished when the HaCaT cells were treated with M5 and the Spiraea prunifolia var. simpliciflora extract at the same time.

From the above results, it has been analyzed that the Spiraea prunifolia var. simpliciflora extract exhibits the effect of treating skin disease induced by IL-6.

### Example 3

### Analysis of Effect of Spiraea prunifolia var. simpliciflora Extract on IL-6-Related Signaling

Through Example 2, it was confirmed that the Spiraea prunifolia var. simpliciflora extract inhibited the production of IL-6, and it is known that one of the main factors of inducing the production of IL-6 is phosphorylation of Stat3 protein.

Therefore, it was attempted to determine whether the Spiraea prunifolia var. simpliciflora extract provided in the present invention was able to inhibit the level of phosphorylated Stat3 protein (pSTAT3).

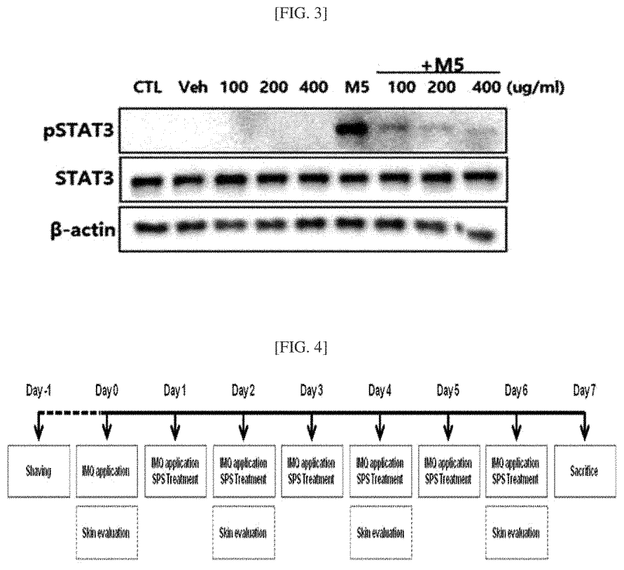

Roughly, the HaCaT cell lines were treated with M5 and the Spiraea prunifolia var. simpliciflora extract at various concentrations (100 μg/mL, 200 μg/mL, or 400 μg/mL) singly or in combination and cultured for 4 hours, and then proteins were extracted from each of these cell lines. Western blot analysis was performed using the proteins extracted from each cell line, anti-pSTAT3 antibody or anti-STAT3 antibody, and anti-rabbit IgG HRP linked antibody to quantitatively analyze the levels of pSTAT3 and STAT3 proteins expressed in each cell line (FIG. 3). Here, β-actin was used as an internal control.

FIG. 3 is a photograph illustrating the Western blot analysis results showing the results acquired by comparing changes in the phosphorylation level of Stat3 protein depending on the concentration of Spiraea prunifolia var. simpliciflora extract treated.

As illustrated in FIG. 3, it was confirmed that the level of phosphorylated Stat3 protein (pSTAT3) significantly increased when the HaCaT cell line was treated with M5 only but the level of pSTAT3 increased by the treatment with M5 was diminished when the cell line was treated with M5 and the Spiraea prunifolia var. simpliciflora extract at the same time.

From the above results, it has been found that the effect of inhibiting the production of IL-6 by the Spiraea prunifolia var. simpliciflora extract is exerted by diminishing the level of phosphorylated Stat3 protein (pSTAT3) that induces the production of IL-6.

### Example 4

### Verification of Effect of Spiraea prunifolia var. simpliciflora Extract Using Animal Model of Skin Disease

Imiquimod (IMQ), which can simulate the symptoms of skin psoriasis, was applied to the skin of mice to obtain an animal model of skin disease. The imiquimod used in the test was Aldara cream (5% imiquimod, 3M Health Care Ltd. UK), and Dermovate (clobetasol 17-propionate 0.5 mg/1 g, Glaxo Operation, UK) was used as a control.

Roughly, the Aldara cream was applied to the depilated ear area of 7-week-old Balb/c mice for 7 days to induce a skin condition similar to skin disease, and the ointment containing the Spiraea prunifolia var. simpliciflora extract was applied together from the 2nd day. The thickness of the ear skin and the level of erythema were each measured when 0, 2, 4, and 6 days had elapsed (FIG. 4). At this time, the thickness of the ear skin was measured using Digimatic Thickness Gauge (Mitutoyo, Japan), and the level of erythema was measured using Dermacatch® (Colorix, Swiss). The ointment containing the Spiraea prunifolia var. simpliciflora extract was prepared by mixing white petrolatum: 4521.6 mg, sorbitan sesquioleate: 23.86 mg, propylene glycol: 204.50 mg, hazel extract (5%): 250 mg, and an appropriate amount of ointment base while boiling the mixture at 50° C. The ointment to be applied to the control was prepared so as to contain white petrolatum, sorbitan sesquioleate, and propylene glycol. Finally, when 5 hours had elapsed after 62.5 mg of Aldara Cream had been applied to the skin of mice, 80 mg of the ointment containing hazel extract or Dermovate was applied.

FIG. 4 is a schematic diagram illustrating the experimental process for verifying the effect of Spiraea prunifolia var. simpliciflora extract using an animal model of skin disease.

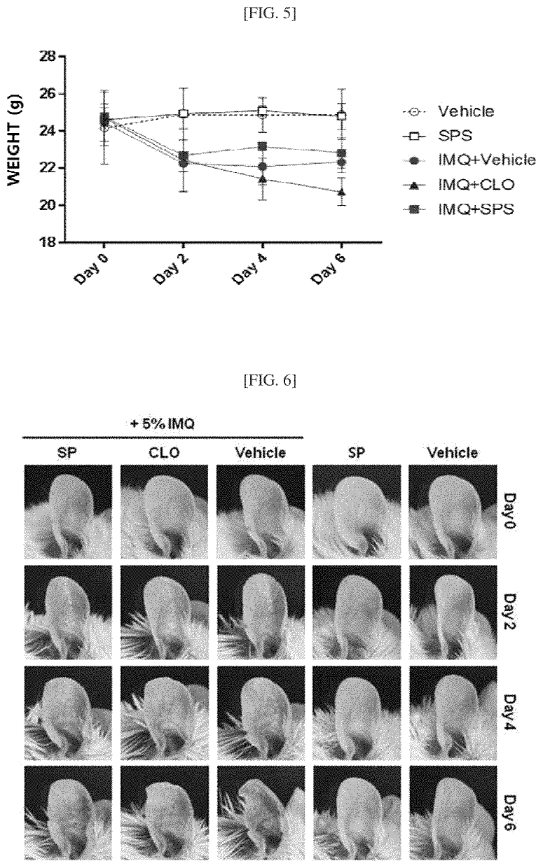

First, changes in the body weight of an animal that was induced to have a skin condition similar to skin disease were measured (FIG. 5). Here, Balb/c mice treated with the ointment for the control were used as a control.

FIG. 5 is a graph illustrating changes in the body weight of Balb/c mice treated with imiquimod Spiraea prunifolia var. simpliciflora extract (SPS), or Dermovate (CLO).

As illustrated in FIG. 5, it was confirmed that the control (vehicle) and mice (SPS) treated only with the Spiraea prunifolia var. simpliciflora extract did not have any significant change in body weight, but all mice (IMQ) treated with imiquimod lost weight, and in particular, mice (IMQ+CLO) treated with imiquimod and Dermovate together had the greatest change in body weight.

Second, changes in the appearance of the ears of mice were visually observed during the experimental process (FIG. 6).

FIG. 6 is a photograph illustrating changes in the appearance of ears of Balb/c mice treated with imiquimod (IMQ), Spiraea prunifolia var. simpliciflora extract (SPS), or Dermovate (CLO) depending on the passage of breeding time.

As illustrated in FIG. 6, when 6 days elapsed, the ears of mice treated with imiquimod (IMQ+Vehicle) only or imiquimod and Dermovate (IMQ+CLO) together had significant changes in the appearance, but no significant change in the appearance was observed from the ears of mice (CH/IMQ+SPS) treated with the Spiraea prunifolia var. simpliciflora extract.

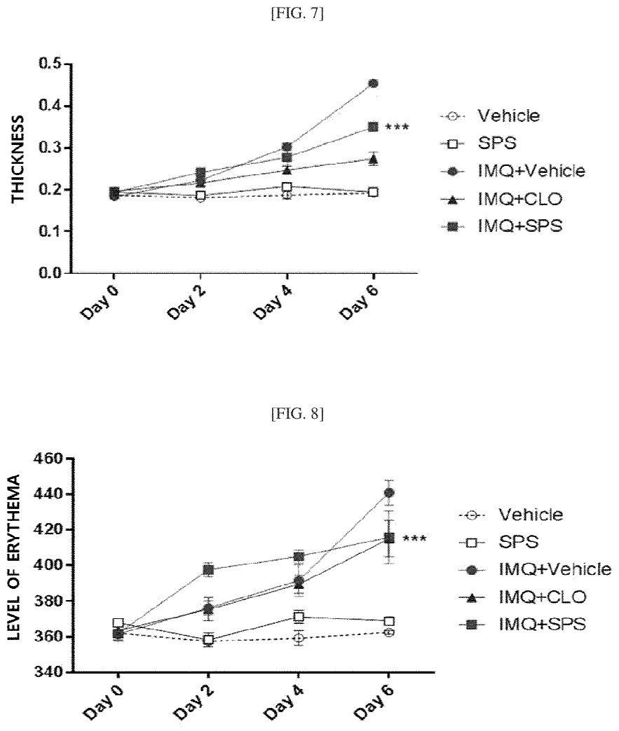

Third, the thicknesses of ear skin of mice were compared with one another during the experimental process (FIG. 7).

FIG. 7 is a graph illustrating changes in the thickness of ear skin of Balb/c mice treated with imiquimod (IMQ), Spiraea prunifolia var. simpliciflora extract (SPS), or Dermovate (CLO) depending on the passage of breeding time.

As illustrated in FIG. 7, it was confirmed that the thickness of ear skin of mice (IMQ+Vehicle) treated only with imiquimod significantly increased as compared to the thicknesses of ear skin of the control (vehicle) and mice (SPS) treated only with the Spiraea prunifolia var. simpliciflora extract, and the thickness of ear skin increased due to the treatment with imiquimod was diminished in mice (IMQ+CLO) treated with imiquimod and Dermovate together and mice (IMQ+SPS) treated with imiquimod and the Spiraea prunifolia var. simpliciflora extract together. It was analyzed that this increase in the thickness of ear skin was caused by an increase in skin keratin.

Fourth, the levels of erythema in the ears of mice were compared with one another during the experimental process (FIG. 8).

FIG. 8 is a graph illustrating changes in the level of erythema of ear skin of Balb/c mice treated with imiquimod (IMQ), Spiraea prunifolia var. simpliciflora extract (SPS), or Dermovate (CLO) depending on the passage of breeding time.

As illustrated in FIG. 8, it was confirmed that the level of erythema of the ear skin of mice (IMQ+Vehicle) treated only with imiquimod significantly increased as compared to the levels of erythema of the ear skin of the control (vehicle) and mice (SPS) treated only with the Spiraea prunifolia var. simpliciflora extract, and the level of erythema of the ear skin, which had increased due to the treatment with imiquimod, was diminished in mice (IMQ+CLO) treated with imiquimod and Dermovate together and mice (IMQ+SPS) treated with imiquimod and the Spiraea prunifolia var. simpliciflora extract together.

Summarizing the results of FIGS. 5 to 8, it has been found that the treatment with the Spiraea prunifolia var. simpliciflora extract can effectively inhibit skin thickening (increased skin keratin) and erythema formation induced by skin disease.

### Example 5

### Verification of Effect of Spiraea prunifolia var. simpliciflora Extract Through Biopsy

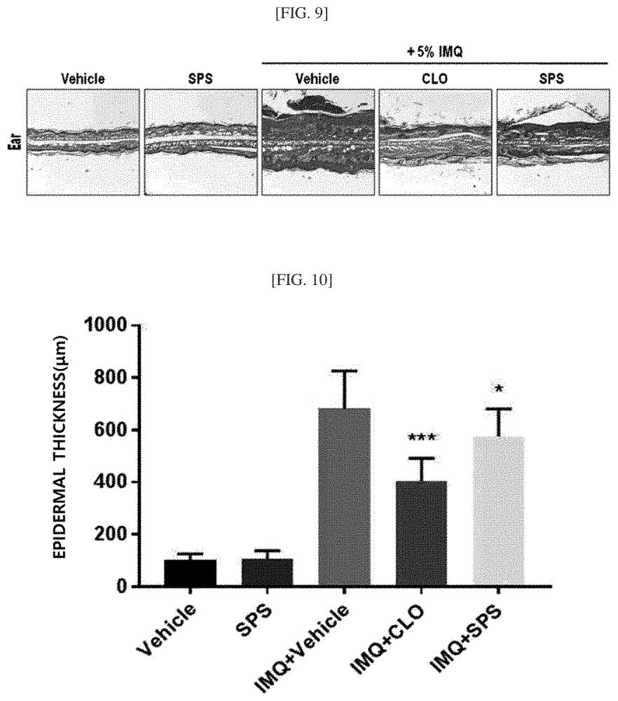

Balb/c mice treated with imiquimod (IMQ), Spiraea prunifolia var. simpliciflora extract (SPS), or Dermovate (CLO) for 7 days in Example 4 were sacrificed, and the skin tissue to which imiquimod, Spiraea prunifolia var. simpliciflora extract, or Dermovate was applied was excised. A slice of the excised tissue was prepared, stained with hematoxylin and eosin (H&E), and then observed under an optical microscope to compare the degree of damage to the skin tissue, and the thickness of the skin epidermis was quantitatively analyzed (FIGS. 9 and 10).

FIG. 9 is an optical micrograph illustrating the results acquired by staining the skin tissue of Balb/c mice treated with imiquimod (IMQ), Spiraea prunifolia var. simpliciflora extract (SPS), or Dermovate (CLO) with hematoxylin and eosin, and FIG. 10 is a graph illustrating the quantitative analysis results of the epidermal thickness of the skin tissue of Balb/c mice treated with imiquimod (IMQ), Spiraea prunifolia var. simpliciflora extract (SPS). or Dermovate (CLO).

As illustrated in FIGS. 9 and 10, it was confirmed that when mice (IMQ+Vehicle) were treated only with imiquimod, the skin thickness was significantly thickened as compared to that of the control (Vehicle), but the level of thickening of the skin thickness was diminished by the treatment with the Spiraea prunifolia var. simpliciflora extract (IMQ+SPS) or Dermovate (IMQ+CLO). It was analyzed that this increase in skin thickness was caused by an increase in skin keratin.

Summarizing the above results, the efficacy of Spiraea prunifolia var. simpliciflora extract was evaluated in vitro and in animal experimental models for hyperplasia and inflammatory response of keratinocytes, which are one of the main symptoms of skin disease, and as a result, the cell proliferation rate of keratinocytes was decreased by the Spiraea prunifolia var. simpliciflora extract, and thus the effect of treating skin disease could be predicted through inhibition of the proliferation of keratinocytes in HaCaT cells, and the production of IL-6 and phosphorylation of Stat3 were confirmed to be significantly diminished, and the possibility of inhibiting inflammation-mediated skin disease induction was confirmed. In an animal model of IMQ-induced skin disease, it was found that the levels of erythema, thickness, and keratin of the skin were diminished by the Spiraea prunifolia var. simpliciflora extract, and it was thus confirmed that the Spiraea prunifolia var. simpliciflora extract directly inhibited the symptoms related to skin disease. Consequently, it is considered that the improvement and treatment efficiency of skin disease can be significantly increased by using the present invention.

From the above description, those skilled in the art to which the present invention pertains will understand that the present invention may be implemented in a different specific form without changing the technical spirit or essential characteristics thereof. Therefore, it should be understood that the embodiments described above are not limitative, but illustrative in all aspects. The scope of the disclosure is defined by the appended claims rather than by the description preceding them, and therefore all changes or modifications derived from the meaning and scope of the following claims and their equivalents should be construed as being included in the scope of the present invention.

1. A method for prevention or treatment of skin disease, comprising administering a composition which comprises Spiraea prunifolia var. simpliciflora extract or a fraction thereof to a subject.

2. The method according to claim 1, wherein the Spiraea prunifolia var. simpliciflora extract inhibits phosphorylation of STAT3 protein to inhibit secretion of inflammatory cytokines.

3. The method according to claim 1, wherein the extract is obtained by extracting any one selected from the group consisting of flowers, leaves, fruits, stems, roots, bark, and sap of Spiraea prunifolia var. simpliciflora, and any combination of the flowers, leaves, fruits, stems, roots, bark, and sap of Spiraea prunifolia var. simpliciflora with one or more solvents selected from the group consisting of water, an alcohol having 1 to 4 carbon atoms, and any mixed solvent of water and an alcohol having 1 to 4 carbon atoms.

4. The method according to claim 1, wherein the fraction is obtained by fractionating Spiraea prunifolia var. simpliciflora extract with a solvent selected from the group consisting of water, an alcohol having 1 to 4 carbon atoms, hexane, ethyl acetate, chloroform, dichloromethane, and any mixed solvent of water, an alcohol having 1 to 4 carbon atoms, hexane, ethyl acetate, chloroform, and dichloromethane.

5. The method according to claim 1, wherein the skin disease is an inflammatory skin disease induced by excessive secretion of inflammatory cytokines.

6. The method according to claim 5, wherein the inflammatory skin disease is a disease selected from the group consisting of atopic dermatitis induced by excessive secretion of IL-6, allergic dermatitis, psoriasis, seborrheic dermatitis, contact dermatitis, lupus erythematosus, purulent acne, papular urticaria, and any combination of atopic dermatitis induced by excessive secretion of IL-6, allergic dermatitis, psoriasis, seborrheic dermatitis, contact dermatitis, lupus erythematosus, purulent acne, and papular urticaria.

7. The method according to claim 1, wherein the composition further comprises a pharmaceutically acceptable carrier, excipient, or diluent.

8-10. (canceled)

11. A method for prevention or improvement of skin disease, comprising applying a composition which comprises Spiraea prunifolia var. simpliciflora extract or a fraction thereof to a subject.
